(19) United States
(12) Patent Application Publication
(10) Pub. No.: US 2026/0365619 A1
(43) Pub. Date: Nov. 19, 2026

(54) METHODS OF BIOMANUFACTURING AND PURIFYING RETINOIDS

(71) Applicant: Amyris Inc., Emeryville, CA (US)

(72) Inventors: Binita Bhattacharjee, Emeryville, CA (US); Thiane Carneiro, Emeryville, CA (US); Annie Preece-Scaringe, Emeryville, CA (US); Benjamin Yap, Emeryville, CA (US)

(21) Appl. No.: 19/107,303

(22) PCT Filed: Sep. 1, 2023
(86) PCT No.: PCT/US2023/031838

§ 371 Date: Feb. 27, 2025

Related U.S. Application Data
(60) Provisional application No. 63/374,274, filed on Sep. 1, 2022.

(51) Int. Cl. C07C 403/08

(52) U.S. Cl. CPC C07C 403/08; C07C 2601/16

(57) ABSTRACT

Provided herein are methods of isolating a retinoid that includes: providing a fermentation broth comprising the retinoid and an overlay; performing a liquid-solid and liquid-liquid separation on the fermentation broth to produce a crude oil and a waste heavy phase; mixing the crude oil with a solvent extraction system to extract the retinoid from the crude oil; and stripping the solvent extraction system from the extracted retinoid to thereby isolate the retinoid. Also provided herein are methods of isolating a retinoid from a composition including the retinoid and farnesol that include: providing a composition including the retinoid and farnesol; feeding the composition into a chromatography system; and collecting the retinoid, thereby isolating the retinoid.

### CROSS-REFERENCE TO RELATED APPLICATIONS

This application claims priority to U.S. Provisional Patent Application No. 63/374,274, filed on Sep. 1, 2022, the entire contents of which is herein incorporated by reference in its entirety.

### TECHNICAL FIELD

This invention relates to methods of biomanufacturing and purifying retinoids.

### BACKGROUND

Retinoids, in particular all-trans retinol, has been widely used in the cosmetic industry for its anti-aging properties and as a supplement to treat vitamin A deficiency. Commercially available retinol has been chemically synthesized. One known limitation of chemically synthesizing retinol is the cost and yield. Fermentation can be a cheaper alternative to chemical synthesis, and provides the opportunity to produce and purify retinol at large scale.

### SUMMARY OF THE INVENTION

Provided herein are methods of isolating a retinoid that include: providing a fermentation broth comprising the retinoid and an overlay; performing a liquid-solid and a liquid-liquid separation on the fermentation broth to produce a crude oil and a waste heavy phase; mixing the crude oil with a solvent extraction system to extract the retinoid from the crude oil; and stripping the solvent extraction system from the extracted retinoid to thereby isolate the retinoid.

In some embodiments, the solvent extraction system comprises ethanol, isopropyl alcohol (IPA), acetone, or propanediol, or a combination thereof. In some embodiments, the solvent extraction system comprises about 70 wt % to about 99.95 wt % ethanol.

In some embodiments, the solvent extraction system comprises acetic acid, acetonitrile, methanol, n-propanol, methyl acetate, ethyl acetate, n-hexane, cyclohexane, or n-heptane.

In some embodiments, the solvent extraction system includes ethanol, wherein the ethanol is free of additives. In some embodiments, the ethanol free of additives is a denatured ethanol.

In some embodiments, the solvent extraction system includes ethanol and isopropyl alcohol. In some embodiments, the solvent extraction system includes between about 90% and about 99% (e.g., about 90%, about 91%, about 92%, about 93%, about 94%, about 95%, about 96%, about 97%, about 98%, or about 99%) ethanol and between about 1% and about 10% (e.g., about 1%, about 2%, about 3%, about 4%, about 5%, about 6%, about 7%, about 8%, about 9%, or about 10%) isopropyl alcohol. In some embodiments, the solvent extraction system includes about 95% ethanol and about 5% isopropyl alcohol.

In some embodiments, the solvent extraction system includes ethanol and n-heptane. In some embodiments, the solvent extraction system includes between about 90% and about 99% (e.g., about 90%, about 91%, about 92%, about 93%, about 94%, about 95%, about 96%, about 97%, about 98%, or about 99%) ethanol and between about 1% and about 10% (e.g., about 1%, about 2%, about 3%, about 4%, about 5%, about 6%, about 7%, about 8%, about 9%, or about 10%) n-heptane. In some embodiments, the solvent extraction system includes about 95% ethanol and about 5% n-heptane.

In some embodiments, the solvent extraction system includes ethanol, methanol, ethyl acetate, and 4-methylpentan-2-one. In some embodiments, the solvent extraction system includes between about 90% and about 100% (e.g., about 90%, about 91%, about 92%, about 93%, about 94%, about 95%, about 96%, about 97%, about 98%, about 99%, or about 100%) ethanol, between about 3% and about 5% (about 3%, about 3.5%, about 4%, about 4.5%, about 4.6%, about 4.8%, about 4.9%, or about 5%) methanol, between about 1% and about 5% (e.g., about 1%, about 1.5%, about 2%, about 2.5%, about 3%, about 3.55, about 4%, about 4.5%, about 4.6%, about 4.8%, about 4.9%, or about 5%)ethyl acetate, between about 1% and about 5% (e.g., about 1%, about 1.5%, about 2%, about 2.5%, about 3%, about 3.55, about 4%, about 4.5%, about 4.6%, about 4.8%, about 4.9%, or about 5%) 4-methylpentan-2-one. In some embodiments, the solvent extraction system includes between about 90% and about 99% ethanol, between about 3% and about 4.5% methanol, between about 1% and about 4% ethyl acetate, and between about 1% and about 4% 4-methylpentan-2-one.

In some embodiments, the solvent extraction system includes between about 90% ethanol, about 3% methanol, about 1%, ethyl acetate, about 1% 4-methylpentan-2-one.

In some embodiments, the solvent extraction system comprises methylene chloride, ethyl ether, chloroform, or carbon tetrachloride.

In some embodiments, the water content in the solvent extraction system is between about 0% to about 60%.

In some embodiments of any of the methods described herein, the mass ratio of the solvent extraction system to crude oil ranges from about 10:1 to about 100:1.

In some embodiments of any of the methods described herein, the mass ratio of the solvent extraction system to crude oil ranges from about 1:1 to about 5:1.

In some embodiments, the overlay comprises a mineral oil and/or a vegetable oil.

In some embodiments, the overlay comprises mineral oil, and the mineral oil is Durasyn® 164 polyalphaolefin or Drakeol® 10.

In some embodiments, the overlay comprises vegetable oil, and the vegetable oil is butter, bisabolol oil, canola oil, castor oil, cocoa butter, coconut oil, cod liver oil, corn oil, cottonseed oil, grape seed oil, jojoba oil, kapok seed oil, linseed oil, olive oil, palm kernel oil, palm oil, patchouli oil, peanut oil, poppyseed oil, rapeseed oil, rice bran oil, safflower oil, soybean oil, sunflower oil, tung oil, walnut oil, or wheat germ oil, or a mixture thereof.

In some embodiments, the liquid-liquid separation comprises centrifugation, filtration with a functionalized membrane, tangential flow filtration, and/or sedimentation. In some embodiments, the stripping the solvent extraction system comprises decanting for about 30 minutes to about 24 hours. In some embodiments, stripping the solvent extraction system comprises evaporation or fractional distillation. In some embodiments, the solvent evaporation is performed in a single step. In other embodiments, the solvent evaporation is performed in multiple steps (e.g., two, three, four, five, or six steps).

In some embodiments, the solvent evaporation includes a rising-film evaporator, a falling-film evaporator, a wiped film evaporation, an agitated thin film, or a plate.

In some embodiments, the solvent evaporation is performed in two-steps. In some embodiments, the solvent evaporation is performed in two-steps with recirculation or without recirculation.

In some embodiments, stripping the solvent extraction system comprises evaporating the solvent at a temperature of less than about 50 °C. In some embodiments, stripping the solvent extraction system comprises evaporating solvent at a temperature of about 50 °C to about 200 °C.

In some embodiments, the method further includes feeding the isolated retinoid into a chromatography system comprising a hydrophobic interaction chromatography resin or a reverse-phase chromatography resin. In some embodiments, the hydrophobic interaction chromatography resin is a hydrophobic interaction chromatography resin with a particle size of about 63 μm to about 150 μm. In some embodiments, the method further includes filtering the isolated retinoid prior to feeding the isolated retinoid into the chromatography system. In some embodiments, the method further includes evaporating solvent at a temperature less than 50 °C.

In some embodiments of any of the methods described herein, the method further includes adding a high oleic oil thereby producing a cosmetic composition. In some embodiments, the high oleic oil is a high oleic organic sunflower oil. In some embodiments of any of the methods described herein, the retinoid is retinol.

Provided herein are methods of isolating a retinoid from a composition comprising the retinoid and farnesol that include: providing a composition comprising the retinoid and farnesol; feeding the composition into a chromatography system; and collecting the retinoid, thereby isolating the retinoid.

In some embodiments, the chromatography system comprises a hydrophobic interaction chromatography resin or a reverse-phase chromatography resin.

In some embodiments, the hydrophobic interaction chromatography resin is a hydrophobic interaction chromatography resin with a particle size of about 63 μm to about 150 μm. In some embodiments, the chromatography resin is Purolite™ PCG600, Mitsubishi Diaion™ HP20SS, Purolite™ PCG1200.

In some embodiments, the method further includes filtering the isolated retinoid prior to feeding the composition into the chromatography system. In some embodiments, the method further includes evaporating solvent at a temperature less than 50 °C. In some embodiments, the method further includes adding a high oleic oil thereby produce a cosmetic composition. In some embodiments, the high oleic oil is a high oleic organic sunflower oil.

Provided herein are compositions including the retinoid produced by any of the methods described herein. In some embodiments, the composition is a liquid or a semi-liquid. In some embodiments, the composition is formulated for oral administration, topical administration, or intravenous administration. In some embodiments, the composition is formulated as a sublingual drop, a gel capsule, or a spray.

Also provided herein are cosmetic compositions and pharmaceutical compositions including the retinoid produced by any of the methods described herein. In some embodiments of any of the pharmaceutical compositions described herein, the pharmaceutical composition includes a pharmaceutically acceptable carrier and/or excipient. In some embodiments of any the pharmaceutical compositions described herein, the pharmaceutically acceptable carrier and/or excipient is an adjuvant.

Provided herein are compositions including a retinoid, wherein the retinoid is produced by a method including: a) culturing a population of host cells that are genetically modified to express one or more enzymes of a retinoid biosynthetic pathway in a culture medium and under conditions suitable for the population of host cells to produce the retinoid, thereby producing a fermentation composition; and b) recovering the retinoid from the fermentation composition, wherein the retinoid is present in the composition with a purity of between 40% w/w and 99.9% w/w (e.g., between 40% w/w and 90% w/w, between 40% w/w and 80% w/w, between 40% w/w and 70% w/w, between 50% w/w and 99.9% w/w, between 50% w/w and 90% w/w, between 50% w/w and 80% w/w, between 50% w/w and 70% w/w, between 60% w/w and 99.9% w/w, between 60% w/w and 90% w/w, between 60% w/w and 80% w/w, between 80% w/w and 99.9% w/w, or between 90% w/w and 99.9% w/w).

Provided herein are methods of isolating a retinoid that include: a) culturing a population of host cells that are genetically modified to express one or more enzymes of a retinoid biosynthetic pathway in a culture medium and under conditions suitable for the population of host cells to produce the retinoid, thereby producing a fermentation composition; and b) recovering the retinoid from the fermentation composition, wherein the retinoid is present in the composition with a purity of between 40% w/w and 99.9% w/w (e.g., between 40% w/w and 90% w/w, between 40% w/w and 80% w/w, between 40% w/w and 70% w/w, between 50% w/w and 99.9% w/w, between 50% w/w and 90% w/w, between 50% w/w and 80% w/w, between 50% w/w and 70% w/w, between 60% w/w and 99.9% w/w, between 60% w/w and 90% w/w, between 60% w/w and 80% w/w, between 80% w/w and 99.9% w/w, or between 90% w/w and 99.9% w/w).

The term “solvent extraction system” refers to one or more component(s) that are used in a process by which a composition comprising a retinoid (e.g., retinol) (e.g., a fermentation broth comprising a retinoid (e.g., a fermentation broth comprising retinol), a liquid comprising a retinoid (e.g., a liquid comprising retinol), or a crude oil containing a retinoid (e.g., a crude oil containing retinol)) is separated, based on physiochemical properties (e.g., solubility or distribution coefficient), into two or more (e.g., 2, 3, or 4, or more) different immiscible phases. In one embodiment, solvent extraction removes retinol from oil (overlay) to the solvent phase. A solvent extraction system may include a single solvent, or a mixture of solvents. Non-limiting examples of solvents include: ethanol, isopropyl alcohol (IPA), acetone, or propanediol, acetic acid, acetonitrile, methanol, n-propanol, methyl acetate, ethyl acetate, n-hexane, cyclohexane, n-heptane, methylene chloride, ethyl ether, chloroform, carbon tetrachloride, and isohexane. The selection of a particular solvent can depend, for example, on its distribution coefficient, selectivity, (in) solubility, recoverability, viscosity, vapor pressure, thermostability, toxicity, allergenic properties, availability, and/or cost.

The term “solvent evaporation” refers to the step(s) in which the solvent is removed from a retinoid (e.g., retinol) by evaporation. Solvent evaporation can be performed in a single step or in multiple steps in series. Non-limiting examples solvent evaporation include use of a tank or tubular, rising-film evaporator (RFE), falling-film evaporator, wiped film evaporation (WFE), agitated thin film, or a plate. Each of these methods of solvent evaporation can be performed as a single pass or multiple passes. In some embodiments, solvent evaporation requires the presence of stream recirculation. In other embodiments, solvent evaporation does not require stream recirculation. In some embodiments, the solvent evaporation occurs in batch mode. In some embodiments, the solvent evaporation occurs in fed-batch mode. In some embodiments, the solvent evaporation is continuous.

The term “clarified fermentation broth” means a fermentation broth obtained from a cell culture that is substantially free of cells (e.g., whole bacteria, or whole yeast cells).

The term “substantially free” refers to a composition (e.g., a purified product) that is at least about 80% free (e.g., at least about 85%, 90%, 95%, 96%, 97%, 98%, or 99% free) of a specified substance (e.g., farnesol).

The term “liquid-liquid separation” is known in the art and refers to any liquid-liquid separation technique that partially or fully separates one or more liquid phases that are immiscible (e.g., one or more oils (e.g., 1, 2, 3 oils) and/or combinations of one or more oils and one or more solvents (e.g., two solvents) from a waste heavy phase. A liquid-liquid separation can demulsify an emulsion (e.g., an emulsion and aqueous phase, an oil and emulsion). Non-limiting examples of liquid-liquid separation include use of a decanter, a solvent, or a combination thereof.

The term “purifying” means performing at least one step to isolate a retinoid (e.g., retinol) from one or more other impurities (e.g., bulk impurities, whole cell debris, and/or farnesol) and/or components present in a liquid that comprises a retinoid (e.g., retinol) (e.g., a clarified fermentation broth comprising a retinoid (e.g., a clarified fermentation broth comprising retinol), or a crude oil), or one or more other components (e.g., DNA, RNA, other proteins, endotoxins, viruses etc.) present in or secreted from cells within a fermentation broth.

The term “filter(ing)” refers to the removal of at least some (e.g., at least 80%, 90%, 95%, 96%, 97%, 98%, or 99%) undesired biological contaminants (e.g., a cell), and/or solvents, from a liquid that comprises a retinoid (e.g., retinol) (e.g., a clarified fermentation broth comprising a retinoid (e.g., retinol), or a crude oil).

The term “eluate” is a term of art and refers to a liquid that exits a chromatography column of a chromatography system that includes a detectable amount of a retinoid (e.g., retinol).

The term “continuous process” means a process that continuously feeds a liquid (e.g., a fermentation broth, a clarified fermentation broth, crude oil, a liquid comprising a retinoid (e.g., retinol), and/or a composition comprising a retinoid (e.g., retinol)) through at least a part of a process. For example, in any of the exemplary continuous processes described herein, a liquid comprising a retinoid (e.g., retinol) (e.g., a fermentation broth, a clarified fermentation broth, crude oil, a liquid comprising a retinoid (e.g., retinol), and/or a composition comprising a retinoid (e.g., retinol)) is continuously fed into the process while it is in operation and an eluate comprising the retinoid (e.g., retinol) is fed out of the process.

The term “closed process” is a term of art that means a process that is performed such that components of the process (e.g., liquid-liquid separation, solvent extraction system, chromatography system) that come into contact with a retinoid (e.g., retinol) are not intentionally exposed to contaminating agents for a significant period of time (e.g., not intentionally air-exposed for a significant period of time).

The term “chromatography system” means a system that includes one or more interconnected or switching chromatography columns and/or chromatographic membranes. A non-limiting example of a chromatography system is simulated moving bed chromatography. Additional examples of chromatography systems are described herein and are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is an exemplary process flow diagram for extraction of retinol.

FIG. 2 shows the chemical structure of retinol.

FIG. 3 shows the chemical structure of farnesol.

FIG. 4A is a chromatogram of gradient elution of a reference solution (1 wt % retinol, 1 wt % farnesol and 0.01 wt % (butylated hydroxytoluene (BHT) in 90/10 ethanol/water solution) using a column packed with Diaion HP20SS (Mitsubishi Chemical). Retinol and farnesol were identified at wavelengths 325 nm and 200 nm, respectively.

FIG. 4B is a chromatogram of gradient elution of a reference solution (1 wt % retinol, 1 wt % farnesol and 0.01 wt % (BHT) in 90/10 ethanol/water solution) using a column packed with Purolite PCG600. Retinol and farnesol were identified at wavelengths 325 nm and 200 nm, respectively.

FIG. 4C is a chromatogram of gradient elution of a reference solution (1 wt % retinol, 1 wt % farnesol and 0.01 wt % (BHT) in 90/10 ethanol/water solution) using a column packed with Purolite PCG950. Retinol and farnesol were identified at wavelengths 325 nm and 200 nm, respectively.

FIG. 5 is an exemplary process flow diagram for isolation of retinol that includes steps of filtration, product purification, solvent evaporation, and blending after solvent extraction.

FIG. 6 is an exemplary process flow diagram showing an exemplary process for retinol extraction. Processes are shown in solid lines (e.g., fermentation, liquid-solid centrifugation, liquid-liquid centrifugation, solvent extraction, evaporation, blend). Streams (e.g., light phase, heavy phase) are shown in dashed lines.

FIG. 7 is a schematic diagram showing one example of solvent extraction of retinol with two feed solutions: (A) Oil feed containing retinol and (B) solvent feed are the inputs and the outputs are (C) the oil layer and (D) the solvent extract. The solvent outlet is the main product, and contains most of the retinol at the end of the process.

FIG. 8A is a graph showing retinol yield (by loss) against ethanol:oil extraction ratios (1, 3, 4.5, and 5) within a glovebox and outside of a glovebox. Moisture and oxygen are removed from the inert atmosphere of a glovebox, which minimizes the amount of moisture that is taken up by the solvent (ethanol). Conditions outside of a glovebox mimic experimental conditions suitable for scale-up manufacturing.

FIG. 8B is a graph showing butylated hydroxytoluene (BHT) yield (by loss) against ethanol:oil extraction ratios (1, 3, 4.5, and 5) within a glovebox and outside of a glovebox.

FIG. 9 is a graph showing BHT:retinol ratio in solvent phase against ethanol:crude oil extraction ratios (1, 3, 4.5, and 5) within a glovebox and outside of a glovebox.

FIG. 10 is a graph showing farnesol/retinol in product [wt %] and retinol yield [%] following twenty two consecutive chromatography runs.

### DETAILED DESCRIPTION

The present disclosure is directed, in part, to methods of isolating a retinoid (e.g., retinol), including, for example, from a fermentation broth or other liquid comprising a retinoid (e.g., retinol) or both a retinoid (e.g., retinol) and farnesol. The methods can include, e.g., providing a fermentation broth comprising a retinoid (e.g., retinol); performing a liquid-liquid separation on the fermentation broth to produce a crude oil and a waste heavy phase; mixing the crude oil with a solvent extraction system to extract the retinoid (e.g., retinol) from the crude oil; and stripping the solvent extraction system from the extracted retinoid (e.g., retinol) to thereby isolate the retinoid (e.g., retinol). Also provided herein are methods of isolating a retinoid (e.g., retinol) from a composition that includes a mixture of a retinoid (e.g., retinol) and farnesol. The methods can include, for example, providing a composition including a retinoid (e.g., retinol) and farnesol; feeding the composition into a chromatography system; and collecting the retinoid (e.g., retinol), thereby isolating the retinoid (e.g., retinol) from the farnesol.

Non-limiting aspects of these methods of these methods are described below. As can be appreciated in the art, the various aspects described below can be used in any combination without limitation.

### Retinol

Retinol or vitamin A alcohol (C₂₀H₃₀O) is the main circulating form of vitamin A found in plasma. Retinol has been shown to be involved in cellular differentiation and proliferation. Retinol or all-trans-retinol is a 20-carbon molecule with a cyclohexenyl ring, a side chain with four double bonds (in trans configuration), and an alcohol end group (FIG. 2). Retinol has a molecular weight of 286.45 and has the following chemical structure:

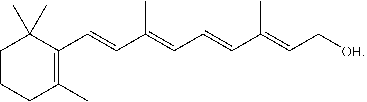 [SMILES: CC1=C(/C=C/C(C)=C/C=C/C(C)=C/CO)C(C)(C)CCC1]

Retinol is commercially available as a crystalline material with a melting point in the 62 °C to 64 °C range. Retinol is known to degrade with exposure to UV, oxygen, and temperature. Exposure to light is preferentially minimized during biomanufacturing, purification and experimentation. Retinol has been used extensively in a variety of antiaging cosmetic products as well as for treatment of skins disorders, such as acne, psoriasis, age spots, discoloration, and melasma. Retinol is used to reduce the effects of stress in skin and sun damage. It can also enhance cell turnover, promote collagen production, facilitate wound healing, and reduce wrinkles and fine lines. Since retinol is only naturally found in animal-derived products, such as milk, cheese, butter, and liver, it is also used as a dietary supplement for vitamin A deficiency (e.g., night blindness and xerophthalmia). Retinol is converted into retinal and retinoic acid.

Overall Process for Isolating A Retinoid from a Fermentation Broth

Provided herein are methods of isolating a retinoid (e.g., retinol). The methods can generally include the steps of providing a fermentation broth including a retinoid (e.g., retinol) and an overlay; performing a liquid-liquid separation on the fermentation broth to produce a crude oil and a waste heavy phase; mixing the crude oil with a solvent extraction system to extract retinoid (e.g., retinol) from the crude oil; and stripping the solvent extraction system from the extracted retinoid (e.g., retinol) to thereby isolate the retinoid (e.g., retinol).

A fermentation broth that includes a retinoid (e.g., retinol) can be generated using any fermentation method employing microorganisms capable of producing a retinoid (e.g., retinol). Methods of genetically modifying microorganisms to produce proteins of interest are known in the art and can be used to modify or cause the expression of enzymes, such as those involved in the production of a retinoid (e.g., retinol), such that large quantities of a retinoid (e.g., retinol) are produced by the microorganisms. In some instances, antioxidants (e.g., BHT) may be added to the fermentation broth to increase the total fermentation efficiency and to prevent the oxidation of a retinoid to another product and other degradation products (e.g., to prevent the oxidation of retinol to retinal and other retinol degradation products).

Processes described herein can include adding an overlay to the fermentation broth. An overlay can, for example, create anaerobic conditions such that a retinoid (e.g., retinol) is not degraded during the isolation process. The overlay can include, e.g., a mineral oil and/or a vegetable oil. Exemplary mineral oils include, e.g., Durasyn® 164 polyalphaolefin or Drakeol® 10 white mineral oil. Exemplary vegetable oils include butter, bisabolol oil, canola oil, castor oil, cocoa butter, coconut oil, cod liver oil, corn oil, cottonseed oil, grape seed oil, jojoba oil, kapok seed oil, linseed oil, olive oil, palm kernel oil, palm oil, patchouli oil, peanut oil, poppyseed oil, rapeseed oil, rice bran oil, safflower oil, soybean oil, sunflower oil, tung oil, walnut oil, or wheat germ oil, or a mixture of at least two of any of such oils.

The liquid-solid separation can be performed using methods known to those of skill in the art. For example, liquid-liquid separation can include centrifugation, filtration with a functionalized membrane (e.g., hydrophobic membranes (e.g., polymeric hydrophobic membranes (e.g., PTFE (polytetrafluoroethylene), PDMS (polydimethyl siloxane), POMS (polyoctylmethyl siloxane) or PEBA (polyether block amide))) and hydrophilic membranes (e.g., Silica-based membranes, zeolite membranes), tangential flow filtration, and/or sedimentation.

Liquid-solid separation can include a step of decanting to separate a liquid from a suspension of insoluble particles and biomass debris (e.g., microorganisms, bacteria, pollutants). For example, in some instances, decanting can be performed for about 30 minutes to about 24 hours (e.g., about 30 minutes to about 3 hours, about 1 hour to about 6 hours, about 1 hour to about 3 hours, or about 1 hour to about 2 hours).

Mixing the crude oil with a solvent extraction system can include, for example, mixing the crude oil with a solvent extraction system for about 5 minutes to about 3 hours (e.g., about 5 minutes to about 45 minutes, about 15 minutes to about 1 hour, or about 15 minutes to about 30 minutes). In some instances, mixing is carried out using rotary agitation. For example, mixing can be performed at a rotary agitation speed of about 300 rotations per minute (RPM) to about 1000 RPM (e.g., about 300 RPM to about 900 RPM, about 300 RPM to about 500 RPM, or about 500 RPM to about 1000 RPM).

A solvent extraction system is used to selectively extract a retinoid (e.g., retinol) from the oil phase of a crude oil containing the retinoid (e.g., retinol). Skilled practitioners will appreciate that a number of different solvent extraction systems can be selected, depending on the desired outcome and the conditions under which the extraction will be performed. The solvent extraction system can include one or more of acetic acid, acetonitrile, methanol, ethanol, acetone, isopropanol, n-propanol, methyl acetate, ethyl acetate, methylene chloride, ethyl ether, chloroform, carbon tetrachloride, n-hexane, isohexane, denatured ethanol, cyclohexane, n-heptane, and mixtures of any two or more of such compositions. In some instances, the solvent extraction system does not include one or more solvents, e.g., in some instances the solvent extraction system may specifically not include methylene chloride, ethyl ether, chloroform, carbon tetrachloride, or a combination thereof. A particularly useful solvent extraction system for use in the presently described methods include ethanol (EtOH), isopropyl alcohol (IPA), acetone, propanediol, or any combination thereof.

In addition to selecting a solvent extraction system, it is important to select the conditions for use of the solvent extraction system to maximize the extraction yield. Both the water content in the solvent/water mixture of the solvent extraction system and the mass ratio of the solvent extraction system to crude oil can play an important role in maximizing extraction yield and will vary depending on the solvent extraction system and overlay. For example, solvent extraction systems described herein can include a water content in the solvent/water mixture of about 0% to about 60% (e.g., about 1% to about 25, about 1% to about 45%, about 1% to about 60%, about 5% to about 45%, about 5% to about 30%, about 10% to about 25%, about 25% to about 60%, or about 30% to about 50%). In some embodiments, the solvent extraction system includes ethanol at about 70 wt % to about 99.95 wt % (e.g., about 70 wt % to about 95 wt %, about 80 wt % to about 99% wt %, about 80 wt % to about 95 wt %, about 90 wt % to about 99% wt %, about 90 wt % to about 95 wt %, or about 90 wt % to about 92 wt %). For example, the mass ratio of any of the solvent extraction systems described herein to crude oil can range from about 10:1 to about 100:1 (e.g., about 10:1 to about 80:1, about 10:1 to about 50:1, about 10:1 to about 20:1, about 20:1 to about 100:1, about 20:1 to about 50:1, about 50:1 to about 100:1, about 70:1 to about 100:1, or about 80:1 to about 100:1). In some instances, the mass ratio of the solvent extraction system to crude oil ranges from about 1:1 to about 5:1 (e.g., about 1:1 to about 4:1, about 1:1 to about 3:1, about 1:1 to about 2:1, about 2:1 to about 4:1, about 3:1 to about 4:1, e.g., about 1:1, about 2:1, about 3:1, about 4:1, or about 5:1).

In order to further separate a retinoid (e.g., retinol) from components found within the solvent extraction system and for future downstream processes and use of the isolated retinoid (e.g., retinol), the solvent extraction system may be stripped. Stripping of the solvent extraction system is useful to separate more than two types of components from each other in an otherwise homogenous mixture. Stripping the solvent extraction system can include evaporation or fractional distillation. For example, stripping the solvent extraction system can include evaporating solvent at a temperature of about 50 °C to about 200 °C (e.g., about 50 °C to about 180 °C, about 50 °C to about 120 °C, about 50 °C to about 100 °C, about 75 °C to about 200 °C, about 75 °C to about 100 °C, about 100 °C to about 200 °C, about 100 °C to about 180 °C, about 100 °C to about 150 °C, about 100 °C to about 120 °C, about 150 °C to about 200 °C, or about 150 °C to about 180 °C). Depending on the solvent, stripping the solvent extraction system can include, e.g., evaporating the solvent at a temperature of less than about 50 °C (e.g., between about 20 °C to about 48 °C, between about 20 °C to about 45 °C, or between about 20 °C to about 30 °C).

Retinol can optionally be separated from farnesol before the solvent system is removed. Farnesol has the following chemical structure:

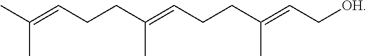 [SMILES: CC(C)=CCC/C(C)=C/CC/C(C)=C/CO]

Because of the chemical similarities between retinol and farnesol, farnesol is not readily separated from retinol using evaporation without resulting in at least some retinol degradation. However, farnesol and retinol can successfully be separated using a chromatography system. Without wishing to be bound by theory, it was found that the isolated retinol, when fed into a chromatography system comprising a hydrophobic interaction chromatography resin (e.g., any of the hydrophobic interaction chromatography resins described herein) or a reverse-phase chromatography resin (e.g., any of the reverse-phase chromatography resins), can selectively be isolated from farnesol, which is a by-product from fermentation and an undesirable impurity for cosmetic applications. Exemplary chromatography resins include Purolite™ PCG600 (polystyrenic macroporous adsorbent non-functionalized resin), Mitsubishi Diaion™ HP20SS (styrene-DVB porous adsorbent resin), Purolite™ PCG1200 (polystyrenic macroporous adsorbent non-functionalized resin).

In some instances, the liquid comprising the isolated retinoid (e.g., retinol) may be filtered prior to feeding the isolated retinoid (e.g., retinol) into a chromatography system. This filtration system removes particulates in the liquid containing the isolated retinoid (e.g., retinol) that could clog or reduce the efficacy of the chromatography resin.

In some instances, a hydrophobic interaction chromatography can be used. A hydrophobic interaction chromatography resin can include a particle size of about 50 μm to about 900 μm (e.g., about 50 μm to about 600 μm, about 50 μm to about 500 μm, about 50 μm to about 200 μm, about 50 μm to about 150 μm, about 50 μm to about 100 μm, about 50 μm to about 80 μm, about 60 μm to about 600 μm, about 60 μm to about 500 μm, about 60 μm to about 200 μm, about 60 μm to about 180 μm, about 60 μm to about 120 μm, about 60 μm to about 100 μm, about 60 μm to about 80 μm, about 100 μm to about 600 μm, about 100 μm to about 500 μm, about 100 μm to about 200 μm, about 100 μm to about 200 μm, about 100 μm to about 150 μm, about 200 μm to about 600 μm, about 200 μm to about 500 μm, or about 500 μm to about 600 μm). The hydrophobic interaction chromatography resins can, for example, have a porosity of about 50 angstrom to about 500 angstrom (e.g., about 50 angstrom to about 200 angstrom, about 50 angstrom to about 100 angstrom, about 75 angstrom to about 250 angstrom about 75 angstrom to about 100 angstrom, about 100 angstrom to about 350 angstrom, about 100 angstrom to about 250 angstrom, about 100 angstrom to about 200 angstrom, or about 100 angstrom to about 150 angstrom).

Skilled practitioners will appreciate that in some instances, methods described herein can further include one or more optional steps of adding one or more components to prepare the retinoid (e.g., retinol) for use in various applications. For example, an antioxidant can be added to the isolated retinoid (e.g., retinol). Such a step can be particularly useful, for example, to make a cosmetic product, as the antioxidant can ameliorate or prevent oxidation of the cosmetic product formulation. Non-limiting examples of antioxidants include: butylated hydroxytoluene (BHT), carnosic acid, Tocobiol® SF ER (a mixture of tocopherol from sunflower oil and vegetable oil), Tocobiol® XT (a mixture of tocopherol with green tea extract), Tocobiol® SF (a mixture of tocopherol from sunflower oil), Tocobiol® ER, Tocobiol® (a mixture of tocopherol from vegetable oil or sunflower oil), alpha-tocopherol, tocopherol, D-alpha-tocopherol acetate, and propyl gallate.

Similarly, skilled practitioners will appreciate that methods described herein can further include adding various components to the isolated retinoid (e.g., retinol), or vice-versa, to produce various products. For example, the isolated retinoid (e.g., retinol) can be added to various other components to produce a cosmetic composition (e.g., essential oils, butters, oils). For example, a high oleic oil (e.g., a high oleic sunflower oil) can be added to the isolated retinoid (e.g., retinol) to produce a cosmetic composition. Exemplary cosmetic compositions include skin creams, lotions, serums, sprays, moisturizers, and the like. As another example, the isolated retinoid (e.g., retinol) can be used to create a pharmaceutical composition, such as a topical composition or an injectable composition to treat various disorders. As yet another example, the isolated retinoid (e.g., retinol) can be formulated to be used as a dietary supplement, or added to various foodstuffs.

Methods of Isolating A Retinoid from a Composition

Provided herein are methods of isolating a retinoid from a composition including a retinoid and farnesol using a chromatography system. The methods can include, for example, providing a composition including a retinoid and farnesol; feeding the composition into a chromatography system (e.g., a chromatography system comprising a hydrophobic interaction chromatography resin (e.g., any of the hydrophobic interaction chromatography resins described herein)); and collecting the retinoid, thereby isolating the retinoid.

Because of the thermal instability of retinol, it is difficult to selectively separate retinol from farnesol, a by-product from fermentation and an undesirable impurity for cosmetic applications. As briefly mentioned above, the structural similarities between retinol and farnesol make their separation even more challenging. However, retinol can be selectively separated from farnesol using a chromatography system. Accordingly, provided herein are methods of isolating retinol from a composition including retinol and farnesol using a chromatography system. The methods can include, for example, providing a composition including retinol and farnesol; feeding the composition into a chromatography system (e.g., a chromatography system comprising a hydrophobic interaction chromatography resin (e.g., any of the hydrophobic interaction chromatography resins described herein)); and collecting retinol, thereby isolating retinol.

In some instances, it may be useful to filter the composition comprising the retinoid (e.g., retinol) and farnesol prior to feeding the composition into a chromatography system. This filtration step can remove particulates in the liquid comprising the composition comprising the retinoid (e.g., retinol) and farnesol that could clog or reduce the efficacy of the chromatography resin.

A chromatography system, as described herein, can include one or more chromatography resins and/or one or more chromatography columns. The chromatography system can include a hydrophobic interaction chromatography resin (e.g., any of the hydrophobic interaction chromatography resins described herein) or a reverse-phase chromatography resin (e.g., any of the reverse-phase chromatography resins). Exemplary chromatography resins include Purolite™ PCG600 (polystyrenic macroporous adsorbent non-functionalized resin), Mitsubishi Diaion™ HP20SS (styrene-DVB porous adsorbent resin), Purolite™ PCG1200 (polystyrenic macroporous adsorbent non-functionalized resin).

Non-limiting examples of hydrophobic interaction chromatography resins useful in the methods described herein can include those having a particle size of about 50 μm to about 900 μm (e.g., about 50 μm to about 600 μm, 50 μm to about 500 μm, 50 μm to about 200 μm, about 50 μm to about 150 μm, about 50 μm to about 100 μm, about 50 μm to about 80 μm, about 60 μm to about 600 μm, about 60 μm to about 500 μm, about 60 μm to about 200 μm, about 60 μm to about 180 μm, about 60 μm to about 120 μm, about 60 μm to about 100 μm, about 60 μm to about 80 μm, about 100 μm to about 600 μm, about 100 μm to about 500 μm, about 100 μm to about 200 μm, about 100 μm to about 150 μm, about 200 μm to about 600 μm, about 200 μm to about 500 μm, or about 500 μm to about 600 μm).

Hydrophobic interaction chromatography resins can have a porosity of about 50 angstrom to about 500 angstrom (e.g., about 50 angstrom to about 200 angstrom, about 50 angstrom to about 100 angstrom, about 75 angstrom to about 250 angstrom about 75 angstrom to about 100 angstrom, about 100 angstrom to about 350 angstrom, about 100 angstrom to about 250 angstrom, about 100 angstrom to about 200 angstrom, or about 100 angstrom to about 150 angstrom).

In some instances, the chromatography system comprises a simulated moving bed chromatography. The simulated moving bed chromatography can be performed continuously over a period of at least 3 days (e.g., at least 7 days, at least 14 days, at least 20 days, at least 60 days, or at least 90 days).

It is well known in the art that a cycle of chromatography can vary depending on the specific biophysical properties of a target of interest (e.g., retinol), the chromatography resin(s), and mobile phases. For example, a hydrophobic chromatography column can include the steps of loading a hydrophobic interaction chromatography column with a fluid including a retinoid (e.g., retinol) and farnesol, washing the column to remove unwanted biological material (e.g., contaminating proteins and/or small proteins), eluting the retinoid (e.g., retinol) bound to the column, and re-equilibrating the column. It is also well-known in the art that any of the single steps in a chromatography cycle can include one or more mobile phases (e.g., two or more mobile phases), and one or more of any of the single steps in a chromatography cycle can include a mobile phase gradient. In some instances, the chromatography system includes two or more (three or more, four or more, five or more, or six or more) cycles of chromatography.

Also provided herein are integrated, closed and continuous processes for isolating a retinoid (e.g., retinol). These processes include providing a composition including a retinoid (e.g., retinol) and farnesol that is substantially free of fermentation broth, cells, cell debris, pollutants, and/or impurities. The process can include continuously feeding a liquid comprising the retinoid (e.g., retinol) and farnesol into a simulated moving bed chromatography system with least one hydrophobic interaction chromatography resin (e.g., Purolite™ PCG600 (polystyrenic macroporous adsorbent non-functionalized resin), Mitsubishi Diaion™ HP20SS (styrene-DVB porous adsorbent resin), Purolite™ PCG1200 (polystyrenic macroporous adsorbent non-functionalized resin), and wherein the process runs continuously from the liquid comprising the retinoid (e.g., retinol) and farnesol to an eluate from the simulated moving bed chromatography that comprises isolated retinoid (e.g., retinol).

The processes described herein provide continuous and time-efficient production of a purified retinoid (e.g., purified retinol) from a liquid including a retinoid (e.g., retinol) and farnesol.

### Compositions and Administration of Compositions

The compositions (e.g., cosmetic compositions, pharmaceutical compositions) including a retinoid produced by any of the methods and processes described herein can be in the form of a liquid, semi-liquid, or solid. The compositions can be formulated for oral administration, topical administration, or intravenous administration. In some instances, the composition is formulated as a sublingual drop, a gel capsule, or a spray. The compositions are administered in a manner compatible with the dosage formulation. A pharmaceutical composition can include a pharmaceutically acceptable carrier and/or an excipient. A pharmaceutical carrier can include any solvents, dispersion media, coatings, adjuvants, stabilizing agents, diluents, preservatives, antibacterial and antifungal agents, isotonic agents, adsorption delaying agents, and the like. In some instances, the composition is a lyophilized composition. Non-limiting examples of adjuvants include aluminum hydroxide, aluminum phosphate, saponins, water-in-oil emulsions, oil-in-water emulsions, and water-in-oil-in-water emulsion. Non-limiting examples of diluents include water, saline, dextrose, ethanol, glycerol, and the like. Non-limiting examples of isotonic agents include sodium chloride, dextrose, mannitol, sorbitol, and lactose.

The invention is further described in the following examples, which do not limit the scope of the invention described in the claims.

### EXAMPLES

### Example 1. Extraction of Retinol from Fermentation Broth

This example demonstrates the effective recovery of retinol from the oil phase by solvent extraction. Skilled practitioners will appreciate that the order of steps described in this example can be modified in certain applications. Accordingly, the method described in the present example is not limiting to the methods of extractions described in the present disclosure.

300 L of whole cell broth and overlay containing 0.61 wt % retinol was subjected to solid-liquid centrifugation and a demulsification step followed by liquid-liquid centrifugation. The recovered light phase was the feed for an extraction step.

Solvent extraction was performed by mixing the crude oil from liquid-liquid centrifugation with a solvent extraction system to selectively extract retinol from the oil phase. Ethanol was used a solvent in these experiments. Screening of conditions for solvent extraction were conducted to determine the recovery yield at varying solvent/oil ratio and ethanol concentrations. For each bench-scale experiment about 10 g of oil and ethanol/water mixture were combined in a 20 mL amber vial at the conditions described in Table 1. Samples were mixed for 30 minutes at 410 rotations per minute (rpm) at ambient temperature (~20 °C), then centrifuged at 3000 rpm for 15 minutes. The resulting oil and solvent layer were assayed for retinol quantification. The highest retinol recovery from oil layer was found with mass ratio 5:1 solvent/oil and ethanol concentrations ≥90 wt %.

TABLE 1
Retinol recovery by varying solvent extraction conditions

| Solvent/Oil | Ethanol content in solvent | Retinol Yield |
|---|---|---|
| Ratio | system (wt %) | (%) |
| 1 | 99.95 | 55 |
| 3 | 99.95 | 80 |
| 5 | 99.95 | 86 |
| 5 | 99.95 | 87 |
| 5 | 95 | 85 |
| 5 | 90 | 82 |
| 5 | 80 | 71 |
| 5 | 75 | 61 |
| 5 | 70 | 51 |

A larger quantity of solvent extraction product was generated by combining 0.5 kg crude oil and 90 wt % ethanol at 5:1 in an amber colored bottle and mixed using a stirring plate at 300-1000 rpm for at least 30 minutes at 20 °C The colored bottled was used to prevent retinol degradation by light exposure. Next, the mixture was transferred to a settling tank to decant for 1-3 hours. Then, the heavy phase, which contained the oil, was drained from the bottom of the vessel and the light phase, containing the ethanol/water and solvent mixture, was collected as a product. Results are shown in Table 2A. The process was scaled to 1.5 kg of crude oil mixed with 90 wt % ethanol at 5:1 in a tank with overhead stirrer (Table 2A).

TABLE 2A
Exemplary experimental results of solvent
extraction at bench and pilot scale

| | | | Farnesol/ | Farnesol/ |
|---|---|---|---|---|
| Solvent | Crude | Retinol | Retinol | Retinol |
| System | oil fed | Yield | in feed | in product |
| [kg] | [kg] | [%] | [wt %] | [wt %] |
| 0.5 | 0.1 | 89 | 1.3 | 1.4 |
| 1.5 | 7.5 | 86 | 1.1 | 0.9 |
| 1.5 | 7.5 | 91 | 1.5 | 1.5 |
| 1.5 | 7.5 | 80 | 1.3 | 1.4 |

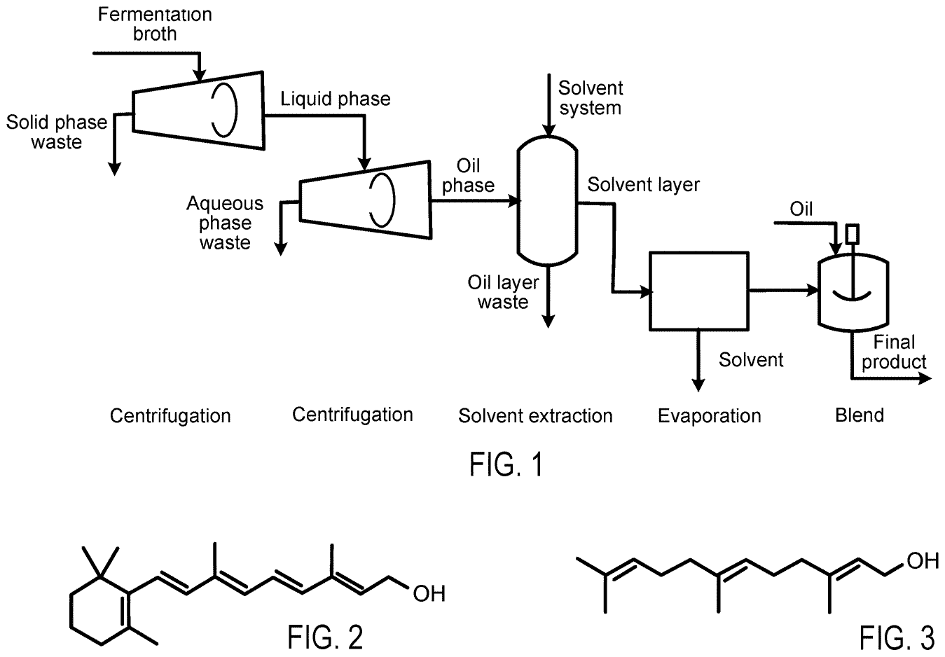

Further retinol purification was achieved by solvent removal by evaporation at low temperature (less than or equal to 50 °C) to avoid retinol degradation. Finally, the evaporation product was blended with sunflower oil to obtain the final desired formulation concentration (FIG. 1).

The farnesol/retinol in feed and farnesol/retinol in product data in Table 2B demonstrate that it is not possible to separate these molecules using this method.

Additional commercial scale data is provided in Table 2B below. These results demonstrate scalability of the process. The same conditions were used in this experiment as for the bench-scale experiment described above: mass oil/mass solvent=1:5 and solvent system was 90:10 ethanol/water.

TABLE 2B
Exemplary experimental results of solvent
extraction at commercial scale

| Crude oil fed [kg] | Retinol Yield [%] |
|---|---|
| 1098 | 82 |
| 1882 | 86 |

### Example 2. Retinol Thermostability by Normal Evaporation

This example demonstrates the thermostability of retinol in crude oil during evaporation.

The crude oil generated from solid-liquid centrifugation and liquid-liquid centrifugation of the fermentation broth and overlay was used as the starting material for the following experiment. Microcentrifuge tubes containing crude oil were heated in a vacuum oven at 70 °C for 1-48 minutes. After each time interval the tube was cooled in an ice bath and the solution was assayed by High-Performance liquid chromatography (HPLC) to determine the peak area percentage of all-trans retinol compared to 9-cis, 11-cis and 13-cis retinol. Results are shown in Table 3. At the start of the experiment (t=0), 97.6% of all-trans retinol was present in the peak area. After 4 minutes at 70 °C, the peak area percentage of all-trans retinol was 96%. After 8 minutes at 70 °C, the peak area percentage of all-trans retinol was 95.1%. After 16 minutes at 70 °C, the peak area percentage of all-trans retinol was 94.1%. After 24 minutes at 70 °C, the peak area percentage of all-trans retinol was 93.1%; and after 48 minutes at 70 °C, the peak area percentage of all-trans retinol was 90.1%.

TABLE 3
Degradation of crude oil all-trans retinol
by normal evaporation at 70 °C over time

| Temperature | Time | All-trans retinol |
|---|---|---|
| (°C) | (min) | (area %) |
| 70 | 0 | 97.6 |
| 70 | 1 | 97.3 |
| 70 | 2 | 97.0 |
| 70 | 4 | 96.0 |
| 70 | 6 | 95.6 |
| 70 | 8 | 95.1 |
| 70 | 16 | 94.1 |
| 70 | 24 | 93.1 |
| 70 | 48 | 90.1 |

### Example 3. Retinol Thermostability by Short-Path Evaporation

This example demonstrates thermostability of retinol during recovery by short path evaporation.

The crude oil generated after solid-liquid centrifugation and liquid-liquid centrifugation of fermentation broth was distilled using a 2″ wiped film evaporation system (Pope Scientific Inc.). This evaporation technique is especially advantageous for heat sensitive molecules because of the shorter residence times. Distillate stream was collected and analyzed by HPLC to determine the peak area percentage of all-trans retinol compared to 9-cis, 11-cis, 13-cis and 9,13-cis retinol. The results are shown in Table 4. After 2.5 minutes at 200 °C and 10 minutes at 200 °C the evaporation product was found to contain 92.8% and 85% peak area all-trans retinol, respectively. After 30 minutes at 200 °C, the evaporation product was found to contain 82.3% peak area all-trans retinol. After 2.5 minutes at 200 °C and 20 minutes at 125 °C the evaporation product was found to contain 98.1% and 94.5% peak area all-trans retinol, respectively. At 50 °C no significant degradation was observed under the time interval of the experiments (i.e., between 0-240 minutes at 50 °C).

TABLE 4
Crude oil all-trans retinol degradation
by short-path evaporation over time

| Temperature | Time | All-trans retinol |
|---|---|---|
| (°C) | (min) | (area %) |
| 200 | 0 | 97.0 |
| 200 | 2.5 | 92.8 |
| 200 | 5 | 90.4 |
| 200 | 10 | 85.0 |
| 200 | 20 | 82.6 |
| 200 | 30 | 82.3 |
| 125 | 0 | 98.8 |
| 125 | 1 | 98.7 |
| 125 | 2.5 | 98.1 |
| 125 | 5 | 97.4 |
| 125 | 10 | 96.7 |
| 125 | 20 | 94.5 |
| 50 | 0 | 98.7 |
| 50 | 15 | 98.6 |
| 50 | 30 | 98.6 |
| 50 | 60 | 98.5 |
| 50 | 120 | 98.5 |
| 50 | 240 | 98.0 |

### Example 4. Solvent Removal by Evaporation

Without wishing to be bound by theory, it was found that by operating evaporation under vacuum at lower temperatures, solvent could be removed without minimizing retinol titer and avoiding all-trans isomer degradation. During these experiments, the temperature ranged from between 0 °C and 40 °C. The pressure ranged between 0.1 torr and 100 torr.

### Conditions

In this study, four different evaporation process configurations were tested-Rotorvap (under batch feeding), Rotorvap (under fed-batch feeding), 2-step evaporation without recirculation (under fed-batch feeding), and 2-step evaporation with recirculation (under fed-batch feeding). The experiments were carried out at bench scale (Rotorvap) and commercial scale (tank). See Table 5.

The study also allowed the testing of different “feeding modes”. According to fed-batch feeding, feed material was systematically added over the course of the run without removing any feed material. According to batch feeding, no feed material was added or removed over the course of the run. The feed mass was divided in small fractions for separate batch evaporations. The concentrated intermediates were then combined for a final evaporation.

### DISCUSSION

As shown in Table 5, there was minimal retinol degradation under all tested conditions. The retinol yield achieved at least 74%. A comparison between the two Rotorvap experiments demonstrated that the less contact the product had with heat, the greater retinol yield (98% retinol yield with Rotorvap with batch fractions as compared to 78% retinol yield with fed-batch feeding). The tested setups were exemplary and the results should not be limited to these setups. Other configurations are feasible and may yield similar results.

TABLE 5
Exemplary experimental results of evaporation at bench and commercial scale

| | | | Feed | | Feed All- | Product All- |
|---|---|---|---|---|---|---|
| | Evaporation set- | Feeding | mass | Retinol | trans | trans |
| Equipment | up | mode | [kg] | yield [%] | isomer % | isomer % |
| Rotorvap | no recirculation | Batch | 8.0 | 98% | 99.2 | 98.9 |
| | | fractions | | | | |
| Rotorvap | no recirculation | Fed-batch | 4.9 | 78% | 98.4 | 98.7 |
| Tank | 2-step | Fed-batch | 4860 | 74% | 99.1 | 98.8 |
| | evaporation, no | | | | | |
| | recirculation | | | | | |
| Tank | 2-step | Fed-batch | 4900 | 81% | 99.1 | 98.5 |
| | evaporation, | | | | | |
| | recirculation | | | | | |

### Example 5. Determination of Solvent Extraction Ratio for Solvent Extraction of Polished Crude Oil

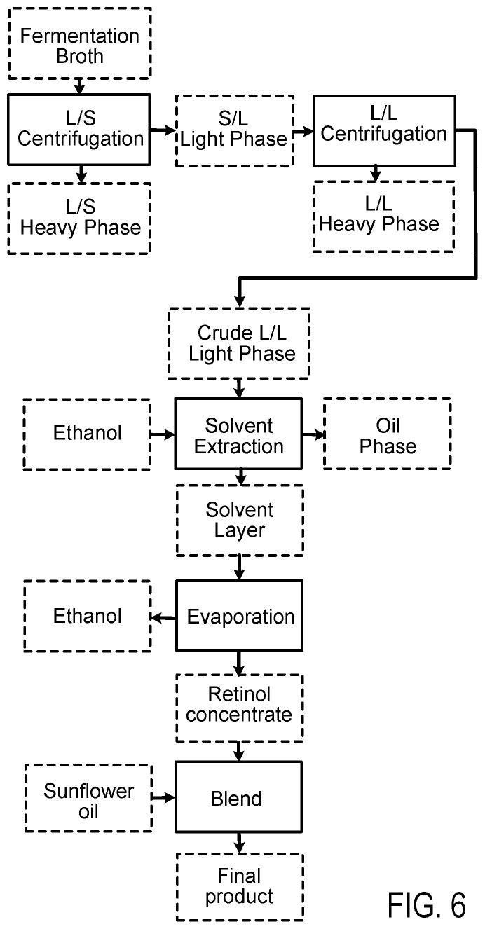

Trans-retinol is known to be thermally labile. Thermal exposure can degrade trans-retinol into cis isomers. In an embodiment, the last step in the production of retinol was evaporation of retinol from polished crude oil in high-oleic sunflower oil (HOSUN). In another embodiment, a process of extracting the retinol into a solvent and then evaporating the solvent off was performed (FIG. 6). It was discovered that a solvent flash could be operated at a much lower temperature than overhead evaporation of retinol, thereby resulting in a much lower thermal degradation of retinol. Use of a wiped film evaporator (WFE) at low temperature could be used to remove solvents from retinol.

### Materials and Methods

Ethanol was tested as the preferred solvent because it is volatile, readily dissolved retinol in concentrated form, exhibited a clean phase separation from HOSUN high oleic sunflower oil, and was globally available in non-genetically modified organism (GMO), kosher, and organic grade. Crude oil, which had been polished by centrifugation and then flashed to reduce water content, was tested as the crude oil feed at three solvent feed:oil extraction ratios: 5:1, 3:1, and 1:1. Experiments were performed first in a glovebox to minimize the amount of moisture taken up in the solvent, and then outside of the glovebox to mimic conditions more suitable for scale-up to manufacturing. Gloveboxes maintain inert atmosphere (less than 1 ppm oxygen and moisture) either by removing the moisture and oxygen from the inert atmosphere of a glovebox or by constant purging of the glovebox with a positive pressure of inert gas. Table 6 outlines the relevant experimental conditions.

TABLE 6
Experimental Conditions

| | | Target Solvent | | |
|---|---|---|---|---|
| | | Feed:Oil Feed | | |
| Test | | Extraction | | Liquid |
| Number | Solvent | Ratio | Solvent Handling | Transfers |
| 3 | Ethanol | 5:1 | Glovebox | Glovebox |
| 4 | Ethanol | 1:1 | Glovebox | Glovebox |
| 5 | Ethanol | 3:1 | Glovebox | Glovebox |
| 6 | Ethanol | 1:1 | Glovebox/Septum | Syringe |
| 7 | Ethanol | 3:1 | Glovebox/Septum | Syringe |
| 8 | Ethanol | 5:1 | Glovebox/Septum | Syringe |

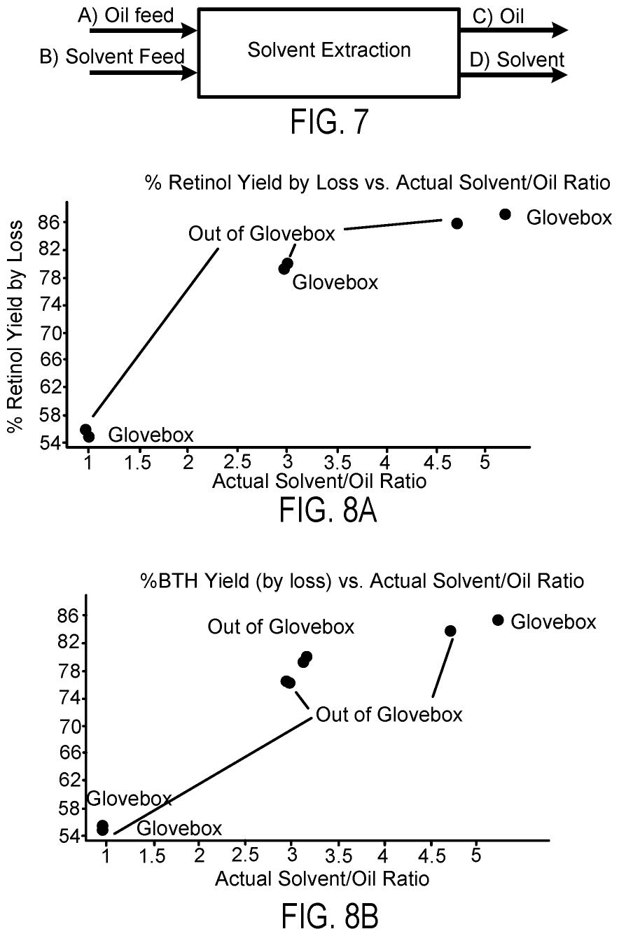

Each retinol extraction was performed in a 20 mL amber scintillation vial with the solvent (ethanol) and crude oil totaling 10 grams. After adding the solvent and crude oil to the vial, the mixture was stirred using a magnetic stir bar on a stir plate with a 20 mL scint vial adapter for 30 minutes at 410 rpm and ambient temperature (~20 °C). After mixing, the mixture was transferred to a centrifuge tube and centrifuged at 3000 rpm for 15 minutes. The four relevant streams for this process are shown in FIG. 7.

Following centrifugation, the oil feed and the solvent feed were both sampled and assayed for retinol quantification, isomer analysis and BHT quantification. A clean phase separation was observed following centrifugation. The solvent layer was removed using a serological pipette and a needle syringe was used to remove the oil layer. All measured data points from Tests 3-8 are provided in Table 7 and the calculated values of interest are shown in Table 8.

TABLE 7
Data Collected from Each Extraction Experiment

| | | | Method of |
|---|---|---|---|
| Data Point | Units | Stream | Measurement |
| Mass | g | Solvent Feed, Oil Feed | Scale/Analytical |
| | | | Balance |
| Retinol (all trans | g/kg | Oil Feed, Solvent, Oil | HPLC |
| retinol + 13 | | | |
| cis retinol) | | | |
| All trans retinol | g/kg | Oil Feed, Solvent, Oil | HPLC |
| All trans retinol | area % | Oil Feed, Solvent, Oil | HPLC |
| Total cis retinol | area % | Oil Feed, Solvent, Oil | HPLC |
| BHT | g/kg | Oil Feed, Solvent, Oil | GC-FID |
| % solids | % | Solvent | Moisture |
| | | | Analyzer |

TABLE 8
Calculated Values of Interest for Each Extraction Experiment

| Value of Interest | Unit | Equation |
|---|---|---|
| Retinol yield (by loss) | % | $\frac{1 - ( {Retinol}_{oil} * \frac{{mass}_{oil}}{1000} )}{{Retinol}_{oil feed} * \frac{{mass}_{oil feed}}{1000}} * 100$ |
| BHT yield (by loss) | % | $\frac{1 - ( {BHT}_{oil} * \frac{{mass}_{oil}}{1000} )}{{BHT}_{oil feed} * \frac{{mass}_{oil feed}}{1000}} * 100$ |
| Retinol Mass Balance | Ratio | $\frac{{Retinol}_{oil} * {mass}_{oil} + {Retinol}_{solvent} * {mass}_{solvent}}{{Retinol}_{oil feed} * {mass}_{oil feed}}$ |
| BHT Mass Balance | Ratio | $\frac{{BHT}_{oil} * {mass}_{oil} + {BHT}_{solvent} * {mass}_{solvent}}{{BHT}_{oil feed} * {mass}_{oil feed}}$ |
| Estimated Retinol Purity (in Solvent- | % | $\frac{{Retinol}_{solvent} * 10}{\% solids} * 100$ |
| Free Extract) | | |
| Estimated BHT Purity (in Solvent- | % | $\frac{{BHT}_{solvent} * 10}{\% solids} * 100$ |
| Free Extract) | | |

### Results

The data collected from these experiments showed that, as the ethanol:feed ratio increased, so did the retinol and BHT yields. FIGS. 8A and 8B showed a positive correlation between ethanol:feed ratio and both retinol and BHT yields. These experiments also showed that yields were comparable between glovebox and out of glovebox handling. FIG. 9 showed a positive correlation between the ethanol extraction ratio and the BHT:retinol ratio, suggesting that a lower extraction ratio was more selective for retinol over BHT. Therefore, a lower extraction ratio was desirable to meet the BHT:retinol specification as defined in Table 8. Experiments performed within the glovebox had a slightly higher starting ratio (0.266) than experiments performed outside of the glovebox (0.259). However, this difference is believed to be negligible for scale-up manufacturing. Because there was little difference between glovebox and out of glovebox handling, it was determined that experiments could be performed at normal atmosphere.

An ethanol extraction ratio of 5 out of the ratios tested resulted in the best retinol yield. Lower extraction ratios tended to increase the retinol/BHT ratio in the extract, but this increased selectivity was not significant enough at this stage to pursue at the expense of retinol yield. A BHT:retinol mass ratio specification of 0.08-0.14 was desirable by reducing the BHT concentration in the fermentation broth and/or tuning the solvent:water composition of the extraction solvent.

Experiments conducts outside of the glovebox did not have a discernible effect on retinol yields, and higher estimated retinol purities were observed with the experiments performed outside of the glovebox.

### Example 6. Impact of Water Content and Solvent/Oil Ratio in Solvent Extraction

To further investigate operating conditions, the impact of water content and solvent/water ratio on solvent extraction yield was studied using crude oils with different retinol concentrations.

### Method

For each set of conditions, crude oil (A or B, containing 171 g/kg and 258 g/kg of retinol, respectively) was mixed with ethanol/water solution (80%, 85%, 90% and 95% ethanol) at a defined ratio solvent:oil (1:1, 5:1 and 9:1) to reach a total mass of 10 g. The mixture was centrifuged at 5000 rpm for 30 minutes. The final solvent and oil layers were recovered and analyzed for retinol titer to calculate process yield.

### DISCUSSION

The results shown in Table 9 demonstrate that higher ethanol concentration in the solvent system is beneficial to the extraction yield. The trend also indicates that increasing the ratio of solvent system to feed oil improves yield, though the extent of that improvement may be limited by costs of solvent consumption by increasing solvent:oil ratio and ethanol %. Results also indicate the impact of feed titer on yield is more significant for conditions in the lower range of ethanol % and solvent:oil ratio tested.

TABLE 9
Extraction yield of retinol [%] from crude oil
for a range of feed titer (A: 171 g/kg retinol, B:
258 g/kg), ethanol/water % and solvent:oil conditions

| Feed | Solvent Solution | solvent:oil ratio | | |
|---|---|---|---|---|
| (Crude oil) | (ethanol/water %) | 1:1 | 5:1 | 9:1 |
| A | 95/5 | 48% | 85% | 95% |
| | 90/10 | 41% | 78% | 88% |
| | 85/15 | 43% | 71% | 86% |
| | 80/20 | 19% | 67% | 85% |
| B | 95/5 | 56% | 87% | 83% |
| | 90/10 | 46% | 82% | 89% |
| | 85/15 | 31% | 65% | 81% |
| | 80/20 | 26% | 74% | 83% |

### Example 7. Testing Different Sources of Ethanol

Ethanol sources may contain additives (e.g., denaturants) that make ethanol unsafe for consumption while still being acceptable for other uses. Undenatured ethanol is an example of an ethanol that is free of additives.

### Method

Crude oil was mixed with 90:10 v/v ethanol/water at a ratio of 1:5 wt oil/solvent. Table 10 provides the list of ethanol solutions that were tested. The total volume of each ethanol solution was approximately 250 mL. First, each ethanol solution was mixed for at least 30 minutes. Next, the ethanol solution was poured into a separation funnel to decant for more than 2 hours. Lastly, the oil layer and the solvent layer were separated and analyzed for retinol concentration.

### DISCUSSION

As shown by the retinol yield obtained in Table 10, denaturants are effective for use in retinol extraction from crude oil. All of the tested ethanol solutions-which varied depending on the mixture of denaturants-had a retinol yield of at least 70%. The highest retinol yield was obtained in an ethanol solution comprising >90% ethanol, between 3-5% methanol, between 1-5% ethyl acetate and between 1-5% 4-methylpentan-2-one.

TABLE 10
Extraction yield of retinol from crude oil using different
sources of ethanol for a 90:10 vol ethanol/water system

| | | Retinol Yield |
|---|---|---|
| Ethanol solution | Composition | [%] |
| 200 proof undenatured | >99% ethanol | 77% |
| ethanol | | |
| Denatured Ethanol | 95% ethanol | 71% |
| SDA 3C | 5% Isopropyl alcohol | |
| Denatured Ethanol | 95% ethanol | 74% |
| CDA 12-A | 5% n-heptane | |
| Denatured Ethanol | >90% ethanol | 79% |
| | >=3-<5% methanol | |
| | >=1-<5% ethyl acetate | |
| | >=1-<5% 4-methylpentan-2- | |
| | one | |

### Example 8. Impurity Removal by Chromatography

This example demonstrates the separation of retinol from farnesol, a C₁₅ sesquiterpene alcohol (C₁₅H₂₆O). Farnesol can be a by-product from fermentation and an undesirable impurity for cosmetic applications. Retinol and farnesol are structurally similar, which makes their separation challenging. Moreover, the thermal instability of retinol limits the possibility of separation of retinol and farnesol by evaporation, as illustrated in Example 2 and Example 3. Chromatography using hydrophobic adsorbents was found to be an efficient and scalable process for retinol/farnesol resolution.

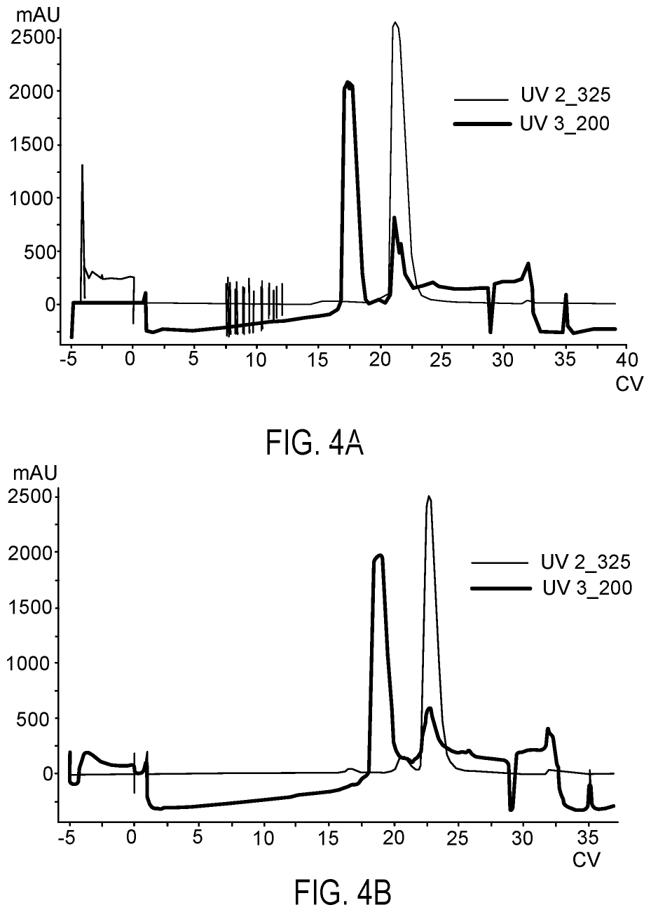

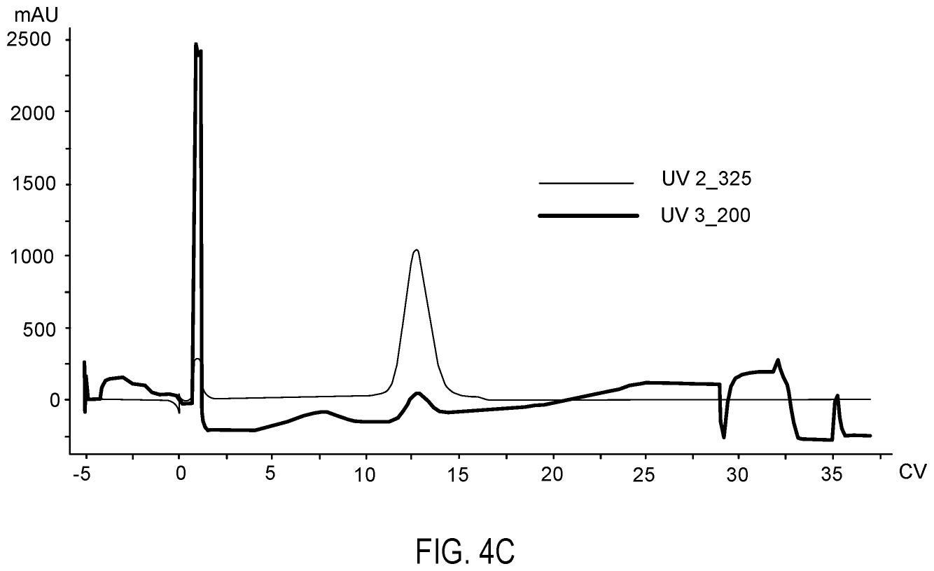

Screening of hydrophobic resins was conducted to evaluate retinol/farnesol separation and guided the choice of process conditions. A 0.5 mL reference solution containing 1 wt % retinol, 1 wt % farnesol and 0.01 wt % butylated hydroxytoluene (BHT) in 90/10 vol % ethanol/water was injected into each column (ID 1.6 cm) packed with 15 g of resin and pre-conditioned at 90/10 vol % ethanol/water. The columns were then washed with 40/60 vol % ethanol/water over 3 bed volumes (BV), eluted with the gradient 40-100 vol % ethanol over 20 BV, and flushed with 100% ethanol over 5 BV. The wash and elution flowrates were set at 2 BV/hour. Retinol and farnesol peaks were identified by absorbance at 325 and 200 nm, respectively. Resulting chromatograms are shown in FIGS. 4A-C. The elution volumes at mid peak are indicated in Table 11 for three exemplary resins, Diaion HP20SS, Purolite™ PCG600, and Purolite™ PCG950, that resulted in clear retinol/farnesol resolution. Hydrophobic adsorbents Diaion™ HP20, Sepabeads™ SP70, Sepabeads™ SP700 and Sepabeads™ SP207 are examples of resins that did not results in efficient retinol/farnesol resolution in the abovementioned conditions.

TABLE 11
Elution of retinol and farnesol from chromatography resins (gradient
elution 40-100 wt % ethanol/water, total volume = 20 BV)

| Chromatography resin | Farnesol mid peak | Retinol mid peak |
|---|---|---|
| Mitsubishi Diaion HP20SS | 17.6 BV | 21.4 BV |
| Purolite ™ PCG600 | 18.8 BV | 22.7 BV |
| Purolite ™ PCG950 | Partially flowthrough, | 12.7 BV |
| | partially 7.6 BV | |

### Example 9. Retinol Purification by Chromatography

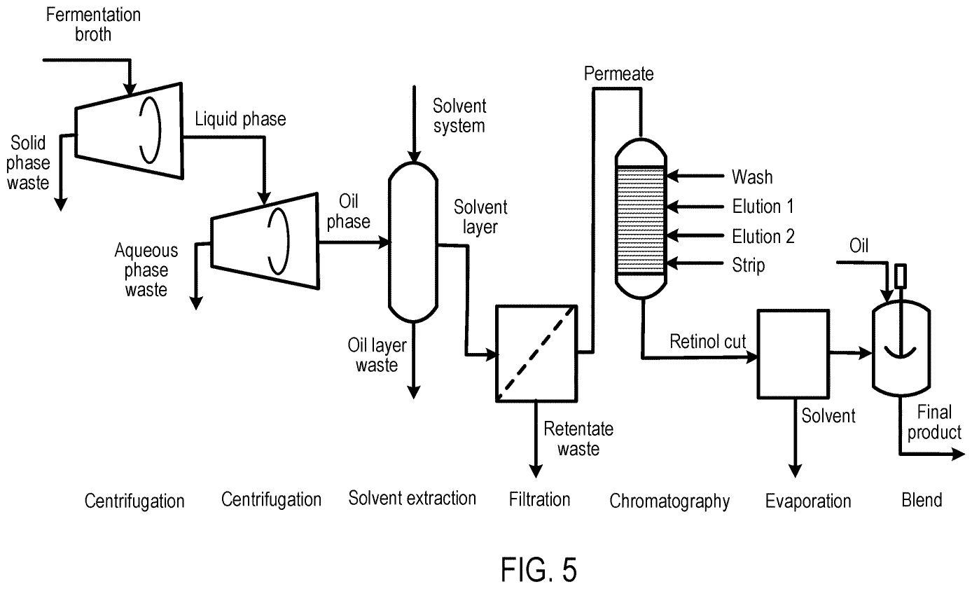

Ethanol solvent extract solutions containing 2.6-3.1 wt % retinol and 1.4-2.4 wt % farnesol/retinol ratio were produced as described in Example 1. The solvent extraction was then followed by a filtration step, a chromatography step for product purification, solvent evaporation and blending steps (FIG. 5).

The solvent layer of the solvent extraction was filtered using a 0.45 microns filter media to remove any remaining cell debris that could impact chromatography performance. The permeate was pumped into a column (ID 1.6 cm) packed with Diaion HP20SS (Mitsubishi Chemical) pre-conditioned at 90/10 vol % ethanol/water. The column was first washed with 5 BV of 70/30 vol % ethanol/water and then eluted with 5 BV of 85/15 vol % ethanol/water and 5 BV of 96/4 vol % ethanol/water. Finally, the column was flushed with 100% ethanol over 5 BV. The elution concentration profiles of both retinol and farnesol were determined by HPLC analysis of chromatography fractions. The product cut, i.e., purified retinol, was selected after elution of farnesol. Two columns with different bed heights were evaluated over a range of resin loadings (0.5-2.5 wt % retinol fed into column/resin). Column details and performance results are shown in Table 12. Surprisingly, given the structural similarity between farnesol and retinol discussed above, little to no farnesol was detected in the product streams, with farnesol/retinol ratios≤0.08 wt %. Even though no particular trend in yield was observed by the increase in loading, the process yielded ≥70% of retinol. While not wishing to be bound by theory, we theorize that by selecting the product cut based on the concentration profile during elution steps rather than a fixed elution step generated the variability in yield. Furthermore, the results show that separation of retinol from farnesol was efficient even at relatively higher loadings (e.g., 2.0 wt % retinol fed/resin and 2.3 wt % retinol fed/resin).

TABLE 12
Retinol yield and purity through chromatography process

| | | Loading | | Purity |
|---|---|---|---|---|
| Resin | Bed | (wt % | Retinol | (farnesol/ |
| mass | height | retinol | yield | retinol |
| (g) | (cm) | fed/resin) | (%) | wt %) |
| 32 | 28 | 0.7 | 83 | 0.07 |
| | | 1.5 | 59 | 0.02 |
| 48 | 45 | 0.5 | 70 | 0.01 |
| | | 0.6 | 90 | 0.02 |
| | | 1.2 | 78 | 0.06 |
| | | 1.5 | 88 | 0.06 |
| | | 1.7 | 74 | 0.02 |
| | | 1.9 | 77 | 0.02 |
| | | 2.0 | 80 | 0.05 |
| | | 2.3 | 93 | 0.07 |
| | | 2.5 | 78 | 0.08 |

The product stream was then evaporated at low temperature and pressures to remove ethanol: 20-40 °C and 0.1-70 torr. After evaporation, the retinol product was blended with high oleic organic sunflower (HOSUN) oil to generate the final retinol product.

The results in Table 13 below demonstrate the scalability of the process.

TABLE 13
Retinol yield and purity through chromatography
process at pilot and commercial scales

| | | | Farnesol/ | Farnesol/ |
|---|---|---|---|---|
| Bed | Bed | Retinol | Retinol | Retinol |
| height | volume | yield | in feed | in product |
| (m) | (L) | (%) | [wt %] | [wt %] |
| 0.4 | 3 | 72% | 1.5 | 0.04% |
| 1.6 | 100 | 60% | 1.5 | 0.12% |
| 1.7 | 1100 | 82% | 1.5 | 0.03% |

### Example 10. Chromatography Reproducibility

In this study, a fixed protocol was repeated over 22 consecutive cycles to verify repeatability.

### Method

The feed was a solvent extraction product containing 2.5 wt % retinol and 1.6 wt % Farnesol/Retinol ratio. The packed chromatography column (bed height 14 cm, bed volume (BV) 28 mL) contained 15 g of Diaion HP20SS resin. The column was equilibrated with 90% ethanol before loading feed solution at 2% retinol/resin. Then the column was washed with 5 BV of 70/30 vol % ethanol/water and the target molecule eluted with 5 BV of 85/15 vol % ethanol/water. Then 5 BV of 95/5 vol % ethanol/water were applied to strip more hydrophobic components. The column was cleaned between each cycle with 5 BVs of isopropyl alcohol.

### Results

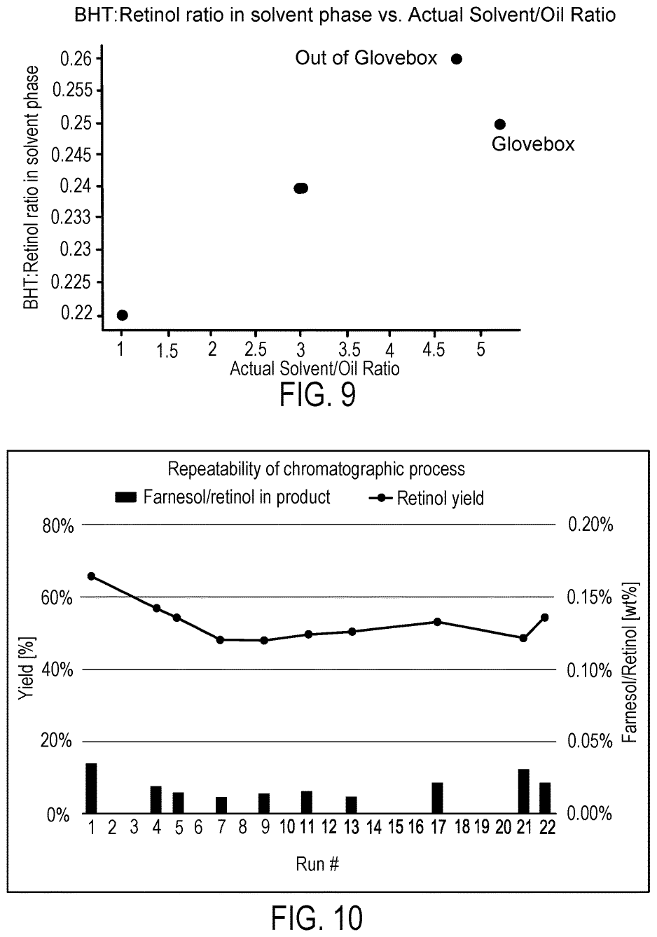

As shown in FIG. 10, the retinol yield dropped between the first 6-7 cycles, which is a common effect for this type of resin. However, the yield became constant and stayed consistent over 22 consecutive cycles without the need for regeneration. The product purity was consistently high, with low amounts of farnesol in the product (below 0.05 wt % Farnesol/Retinol). See also FIG. 10.

### Varying Wash and Elution Conditions

The process may vary in wash, elution and strip conditions for different yield, purity and solvent consumption.

### Method

The feed was a solvent extraction product containing 2 wt % retinol and 1.9 wt % Farnesol/Retinol ratio. The packed chromatography column (bed height 24 cm, bed volume (BV) 48 mL) contained 23 g of Diaion HP20SS resin. The column was equilibrated with 90% ethanol before loading feed solution at 2% retinol/resin. Wash, elution and strip steps conditions are described in Table 14. The column was cleaned after each cycle with 5 BVs of isopropyl alcohol.

TABLE 14
Exemplary experimental results for farnesol removal
by varying wash and elution conditions

| | Wash | | Elution | | Strip | | | |
|---|---|---|---|---|---|---|---|---|
| | [Ethanol | | [Ethanol | | [Ethanol | | | Farnesol/ |
| | % in | Wash | % in | Elution | % in | Strip | | Retinol |
| | ethanol/ | Total | ethanol/ | Total | ethanol/ | Total | Retinol | in |
| Run | water | volume | water | volume | water | volume | Yield | product |
| # | solvent] | [BV] | solvent] | [BV] | solvent] | [BV] | [%] | [wt %] |
| 1 | 70% | 5 | 85% | 5 | 95% | 5 | 82% | 0.16% |
| 2 | 75% | 5 | 85% | 5 | 95% | 5 | 52% | 0.05% |
| 3 | 70% | 5 | 95% | 5 | 95% | 5 | 83% | 0.16% |
| 4 | 80% | 2 | 95% | 5 | 95% | 3 | 65% | 0.02% |
| 5 | 75% | 3 | 95% | 5 | 95% | 3 | 75% | 0.01% |
| 6 | 70% | 5 | 95% | 5 | — | — | 69% | 0.15% |

### DISCUSSION

As shown by the data in Table 14, the varying wash and elution conditions greatly enhanced the retinol yield. Increasing the wash strength (higher ethanol %) resulted in better purity, but led to a lower retinol yield.

The most relevant steps in this process were the wash and elution steps. Farnesol is eluted during the wash step and retinol is eluted during the elution step. Little retinol or farnesol are seen in the strip step. The strip step is useful to strips out more hydrophobic components. Though it seemed like removing the strip step resulted in lower yield (comparison of Runs 3 and 6), the difference in retinol yield was attributed to the use of the resin (HP20SS), i.e. running consecutive cycles in this column, and lower total mass balance recovered rather than retinol lost in the strip step.

### Antioxidant

Chromatography process can partially or totally separate the antioxidant (e.g. BHT, Tocobiol®, or Tocobiol® SF ER) from retinol. To avoid oxidative degradation, an antioxidant (e.g., BHT, Tocobiol®, or Tocobiol® SF ER, or any of the antioxidants described herein) can be added to the chromatography product.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of isolating a retinoid, the method comprising:
providing a fermentation broth comprising the retinoid and an overlay;
performing a liquid-solid and a liquid-liquid separation on the fermentation broth to produce a crude oil and a waste heavy phase;
mixing the crude oil with a solvent extraction system to extract the retinoid from the crude oil; and
stripping the solvent extraction system from the extracted retinoid to thereby isolate the retinoid.

2. The method of claim 1, wherein the solvent extraction system comprises ethanol, isopropyl alcohol (IPA), acetone, or propanediol, or a combination thereof, optionally wherein the solvent extraction system comprises about 70 wt % to about 99.95 wt % ethanol.

3. (canceled)

4. The method of claim 1, wherein the solvent extraction system comprises acetic acid, acetonitrile, methanol, n-propanol, methyl acetate, ethyl acetate, n-hexane, cyclohexane, or n-heptane.

5. The method of claim 2, wherein the solvent extraction system comprises ethanol, wherein the ethanol is free of additives, optionally wherein the ethanol free of additives is a denatured ethanol.

6. (canceled)

7. The method of claim 1, wherein the solvent extraction system comprises (i) ethanol and isopropyl alcohol, (ii) ethanol and n-heptane, (iii) ethanol, methanol, ethyl acetate, and 4-methylpentan-2-one, or (iv) methylene chloride, ethyl ether, chloroform, or carbon tetrachloride.

8.-13. (canceled)

14. The method of claim 1, wherein the overlay comprises a mineral oil and/or a vegetable oil, optionally wherein the overlay comprises vegetable oil, and the vegetable oil is butter, bisabolol oil, canola oil, castor oil, cocoa butter, coconut oil, cod liver oil, corn oil, cottonseed oil, grape seed oil, jojoba oil, kapok seed oil, linseed oil, olive oil, palm kernel oil, palm oil, patchouli oil, peanut oil, poppyseed oil, rapeseed oil, rice bran oil, safflower oil, soybean oil, sunflower oil, tung oil, walnut oil, or wheat germ oil, or a mixture thereof.

15. (canceled)

16. (canceled)

17. The method of claim 1, wherein the liquid-liquid separation comprises centrifugation, filtration with a functionalized membrane, tangential flow filtration, and/or sedimentation.

18. (canceled)

19. The method of claim 1, wherein stripping the solvent extraction system comprises a solvent evaporation or fractional distillation, optionally wherein: the solvent evaporation is performed in a single step, or the solvent evaporation is performed in multiple steps, or the solvent evaporation comprises a rising-film evaporator, a falling-film evaporator, a wiped film evaporation, an agitated thin film, or a plate.

20.-24. (canceled)

25. The method of claim 1, wherein stripping the solvent extraction system comprises evaporating the solvent at a temperature of less than about 50 °C or at a temperature of about 50 °C to about 200 °C.

26. (canceled)

27. The method of claim 1, wherein the method further comprises feeding the isolated retinoid into a chromatography system comprising a hydrophobic interaction chromatography resin or a reverse-phase chromatography resin, optionally wherein the hydrophobic interaction chromatography resin is a hydrophobic interaction chromatography resin with a particle size of about 63 μm to about 150 μm.

28. (canceled)

29. The method of claim 27, wherein the method further comprises filtering the isolated retinoid prior to feeding the isolated retinoid into the chromatography system.

30. The method of claim 29, wherein the method further comprises evaporating solvent at a temperature less than 50 °C.

31. The method of claim 27, wherein the method further comprises adding a high oleic oil.

32. The method of claim 31, wherein the high oleic oil is a high oleic organic sunflower oil.

33. The method of claim 1, wherein the retinoid is retinol.

34. A method of isolating a retinoid from a composition comprising the retinoid and farnesol, the method comprising:
providing a composition comprising the retinoid and farnesol;
feeding the composition into a chromatography system; and
collecting the retinoid, thereby isolating the retinoid.

35. The method of claim 34, wherein the chromatography system comprises a hydrophobic interaction chromatography resin or a reverse-phase chromatography resin.

36. The method of claim 35, wherein the hydrophobic interaction chromatography resin is a hydrophobic interaction chromatography resin with a particle size of about 63 μm to about 150 μm.

37. The method of claim 34, wherein the chromatography resin is Purolite™ PCG600, Mitsubishi Diaion™ HP20SS, Purolite™ PCG1200, or the method further comprises filtering the isolated retinoid prior to feeding the composition into the chromatography system, or the method further comprises evaporating solvent at a temperature less than 50 °C.

38. (canceled)

39. (canceled)

40. The method of claim 37, wherein the method further comprises adding a high oleic oil, optionally wherein the high oleic oil is a high oleic organic sunflower oil.

41.-50. (canceled)
